(19) United States
(12) Patent Application Publication
(10) Pub. No.: US 2012/0101557 A1
(43) Pub. Date: Apr. 26, 2012

(54) TREATMENT APPARATUS AND USE THEREOF FOR TREATING PSORIASIS

(75) Inventors: Giovanna Wagenaar Cacciola, Eindhoven (NL); Matthias Born, Geldren (DE); Christoph Viktor Suschek, Aachen (DE)

(73) Assignee: Koninklijke Philips Electronics N.V., Eindhoven (NL)

(21) Appl. No.: 13/380,565

(22) PCT Filed: Jun. 21, 2010
(86) PCT No.: PCT/IB10/52779

§ 371 Date: Dec. 23, 2011

(30) Foreign Application Priority Data
Jun. 24, 2009 (EP) 09163555.7

(51) Int. Cl. A61N 5/06

(52) U.S. Cl. 607/94; 607/90

(57) ABSTRACT

A treatment apparatus (10) is used for treatment of parts of a skin (5). The apparatus comprises a radiation source (1) emitting radiation, and a radiator (2) for guiding the emitted radiation to the parts of the skin (5). The parts of the skin (5) comprise skin cells affected by psoriasis. The radiation source (1) emits radiation in a first wavelength range of 400-460 nm, in a second wavelength range of 600-700 nm, or in a first wavelength range of 400-460 nm and a second wavelength range of 600-700 nm.

### FIELD OF THE INVENTION

The present invention relates to a treatment apparatus for treatment of parts of a skin, e.g. using irradiation with light.

### BACKGROUND OF THE INVENTION

American patent publication US20020173833 discloses an apparatus for treatment of a skin disorder, the apparatus including a light source having a spectral emittance concentrated in one specific narrow spectral band in the range of 400 to 450 nm (blue light).

### SUMMARY OF THE INVENTION

According to the present invention, a treatment apparatus according to the preamble defined above is provided, in which the treatment apparatus comprises a radiation source emitting radiation and a radiator (mirror/lens/holder for LED's) for guiding the emitted radiation to the parts of the skin, wherein the parts of the skin comprise skin cells affected by psoriasis, and wherein the radiation source emits radiation in a first wavelength range of 400-460 nm, in a second wavelength range of 600-700 nm, or in a first wavelength range of 400-460 nm and a second wavelength range of 600-700 nm. The radiator can have the form of a mirror or lens arrangement, or a holding arrangement for the radiation source allowing directing the emitted radiation. This treatment apparatus allows direct irradiation of psoriatic cells in the skin, which has proven to be very efficient.

In a further aspect, the present invention relates to the use of a treatment apparatus comprising a radiation source emitting radiation and a radiator for guiding the emitted radiation for the treatment of psoriasis, comprising irradiating parts of a skin having skin cells affected by psoriasis, wherein the radiation comprises radiation in a first wavelength range of 400-460 nm, in a second wavelength range of 600-700 nm, or in a first wavelength range of 400-460 nm and a second wavelength range of 600-700 nm. The treatment of psoriasis in this manner has proven to be very efficient, while being more comfortable to the patient than known treatments.

In an embodiment, the radiation treatment as described above is applied in combination with a Psoralen UVA therapy. In a further embodiment, the radiation treatment as described above is applied in combination with a peroxide treatment or UV (UV-A or UV-B) treatment. E.g. irradiation with blue light is applied before a Psoralen UVA or other treatment step, as the blue light increases the susceptibility of the cells to the subsequent toxic insults of peroxide or UVA radiation. As an alternative, the two different treatment may be applied simultaneously.

### BRIEF DESCRIPTION OF THE DRAWINGS

The present invention will be discussed in more detail below, using a number of exemplary embodiments, with reference to the attached drawings, in which

FIG. 1 depicts a schematic diagram of a treatment apparatus according to an embodiment of the present invention;

FIG. 2 depicts a graph showing experimental results of treatment of psoriasis with a treatment apparatus according an embodiment of the present invention;

FIG. 3 depicts a number of graphs showing efficient treatment of psoriasis using a further embodiment of the treatment apparatus according to the present invention;

FIG. 4 depicts a schematic view of a gantry type embodiment of the present treatment apparatus;

FIG. 5 depicts a schematic view of a handheld type embodiment of the present treatment apparatus;

FIG. 6 depicts a schematic view of a plaster type embodiment of the present treatment apparatus; and

FIG. 7 depicts a graph showing the effects of blue light on H₂O₂—or of UVA—induced cell death of human skin fibroblasts.

### DETAILED DESCRIPTION OF EMBODIMENTS

The present invention embodiments are related to an apparatus and use of such an apparatus for treatment of psoriasis. The various embodiments may be adapted for home use or professional use in e.g. a hospital.

Psoriasis is a chronic, non-infectious inflammatory skin disease characterized by well-demarcated plaques where the cells divide (reproduce) quicker than normal, leading this to a very dry and red skin. The proliferative rate of the epidermis is controlled by the combination of the growth fraction and cell cycle time. In the normal skin the number of cells produced is balanced by the number of cells leaving the epidermal proliferative pool. The time required for a cell to transit between the basal layer and the stratum corneum of the skin (basically from birth to death and getting loose from the skin) is about 14 days, while in psoriasis patients is much shorter (about 4 days).

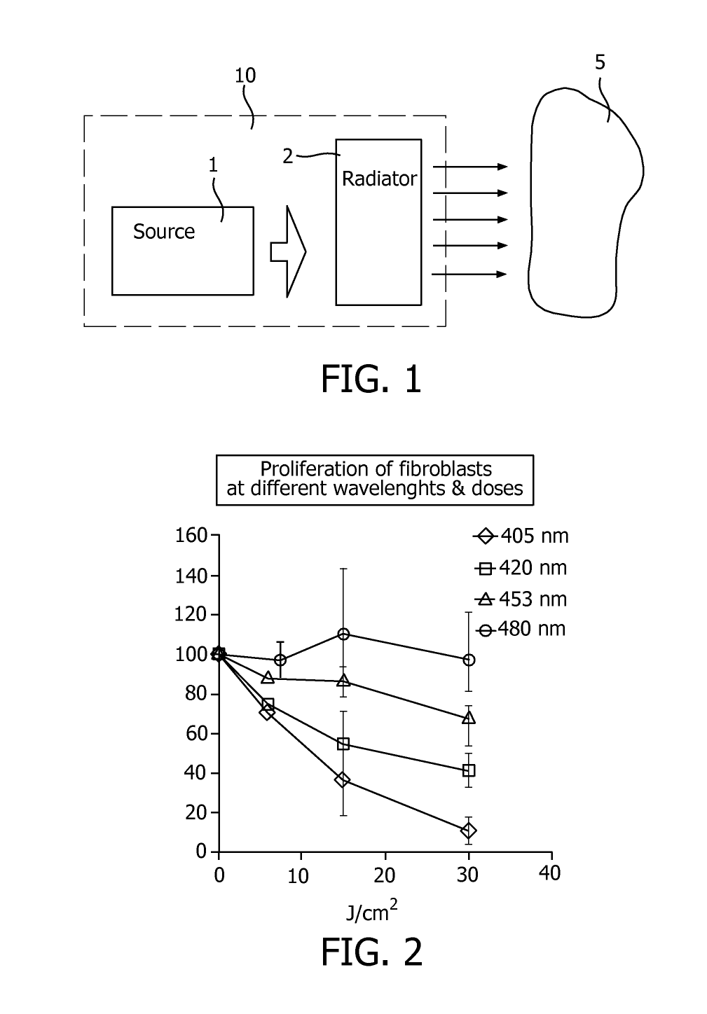

According to an embodiment of the present invention, an apparatus 10 for treatment of parts of a skin 5 is provided as depicted schematically in FIG. 1. The apparatus 10 comprises a radiation source 1 emitting radiation and a radiator 2 (e.g. in the form of a mirror arrangement, lens arrangement or a holder arrangement for the radiation source) for guiding the emitted radiation to the parts of the skin 5, wherein the parts of the skin 5 comprise skin cells affected by psoriasis. Such an apparatus 10 allows direct irradiation of psoriatic cells in affected parts of the skin 5, which has proven to be an efficient treatment.

The radiation source 1 is in further embodiments formed by LED based device, e.g. having a plurality of LED's which emit light in the blue spectrum, red spectrum or blue and red spectrum.

In a first embodiment, the radiation source 1 emits radiation in a first wavelength range of 400-460 nm, e.g. in a range of 410-430 nm. Also, tests have been performed with a radiation source 1 emitting light around 420 nm, which will be described in more detail below. This blue spectrum light has proven to be very effective in the treatment of psoriatic cells of the skin 5, especially when sufficient energy reaches the skin 5. In a further embodiment, the radiator 2 provides an energy in the first wavelength range to the affected parts of the skin 5 of up to 200 mW/cm², e.g. 100 mW/cm^{2.}

In a second embodiment, the radiation source 1 emits radiation in a second wavelength range of 600-700 nm, e.g. with a wavelength of about 630 nm. The treatment of psoriasis with this red range of visible light has also proved to be an effective treatment as will be discussed below with reference to in vivo tests. In these embodiments, treatment where the radiator 2 provides an energy in the second wavelength range to the affected parts of the skin 5 of up to 300 mW/cm² has proven very effective. Also a treatment with an energy of up to 200 mW/cm² has proven to be effective.

In an even further embodiment, the radiation source 1 emits radiation in a first wavelength range of 400-460 nm and in a second wavelength range of 600-700 nm. The combined treatment with both blue and red light has proven to be very effective. It is expected that the combination of blue light which penetrates only lightly in the skin 5 and red light which penetrates deeper into the skin 5 results in the more efficient (direct) treatment of psoriatic cells in the skin 5.

### EXAMPLES

A number of radiation sources 1 have been tested in vitro (with human skin cells in culture). A blue LED device (which is known to be used for anti-acne treatment) has been also tested and proved to be non toxic up to a dose of 200 J/cm2, as the number of live cells after the irradiation with blue light remains more or less unchanged. Results show that repetitive irradiation of the skin 5 with blue light reduces cell division of human skin-derived fibroblasts. A dose-dependent reduction in cell proliferation is observed, as shown in FIG. 2 for various wavelengths. It is shown that the irradiation with light having a wavelength of 453 nm, 420 nm, and 405 nm provide an increasing effectiveness, while irradiation with 480 nm has virtually no effect.

Thus, by tuning the wavelength, irradiation power and exposure time, cell proliferation can be controlled.

Current light therapies for psoriasis are UVB narrow band (312 nm), or UVA combined with psoralen (also called PUVA therapy), an agent which increases the biological UVA-induced effects on skin cells. The systems used for these types of therapy are big devices for full body or partial body treatment. The therapeutic benefit of the PUVA therapy is due to a decrease in the cell growth of hyper-proliferating keratinocytes in psoriatic skin plaques and/or to induced cell death of hyper-reactive T-cells, which are thought to represent the driving force in the pathogenesis of psoriasis, within psoriatic skin lesions. Unfortunately, increased exposure to UVA during PUVA is associated with increased risk for skin cancer, and premature aging of the skin.

Thus, in one embodiment of the present invention, the use of an apparatus 10 emitting blue light achieves a reduction in cell growth of hyper-proliferating keratinocytes in the absence of the deleterious effects of UVA radiation. Furthermore, as blue light increases the susceptibility of cells to the toxic effects of UVA, use of for example royal blue (455 nm) radiation prior to PUVA therapy helps to reduce the therapeutically needed UVA dose and thus, could help to prevent from injurious effects of UVA. Finally, by increasing the susceptibility of cells to the toxic insults of hydrogen peroxide or UVA, a combination of blue light plus H₂O₂ or UVA could be used as a novel therapy approach in the treatment of psoriasis.

In further embodiments of the present invention, a combination of treatment with blue light radiation (400-460 nm) and UVA is used, blue light radiation (400-460 nm) and UVB, red light radiation (600-700 nm) and UVA, or red light radiation (600-700 nm) and UVB. In these methods, the blue or red radiation may be applied simultaneously or consecutively with the UVA or UVB radiation, respectively. For instance, first blue radiation is used as a preparation and then UVB radiation, or first red radiation and then UVB radiation. Although (in a specific exemplary test case) royal blue radiation (455 nm) has proven not to be toxic for the cells, irradiation of human skin fibroblasts with 30 J/cm² royal blue radiation significantly enhances the susceptibility of the cells to the toxic insults of the pro-oxidant agent hydrogen peroxide (H₂O₂) or of UVA/ UVB radiation. As shown in the graph of FIG. 7, pre-irradiation of fibroblast cultures with royal blue radiation followed by the toxic stimulus yielded significantly higher toxicity than seen after hydrogen peroxide- or UVA/UVB-challenge alone. Similar results are expected for use of irradiation in general in the first (blue) wavelength range of 400-460 nm, e.g. at 420 nm.

A study on skin cells indicate that blue light (400-460 nm) slows down the cell proliferation without inducing DNA damage. The studies comprise the measurement of biological actions on human skin cells using different wavelengths, irradiation doses, and irradiation algorithms.

Clinical trials were carried out with 20 patients who were treated on two similar psoriatic plaques on their skin 5. One plaque was irradiated with red light (630 nm) and one with blue light (420 nm). The power density of the blue light irradiation of the skin 5 was ˜90 mW/cm2, the power density of the red light irradiation ˜40 mW/cm2. The red light irradiation was meant as a placebo test.

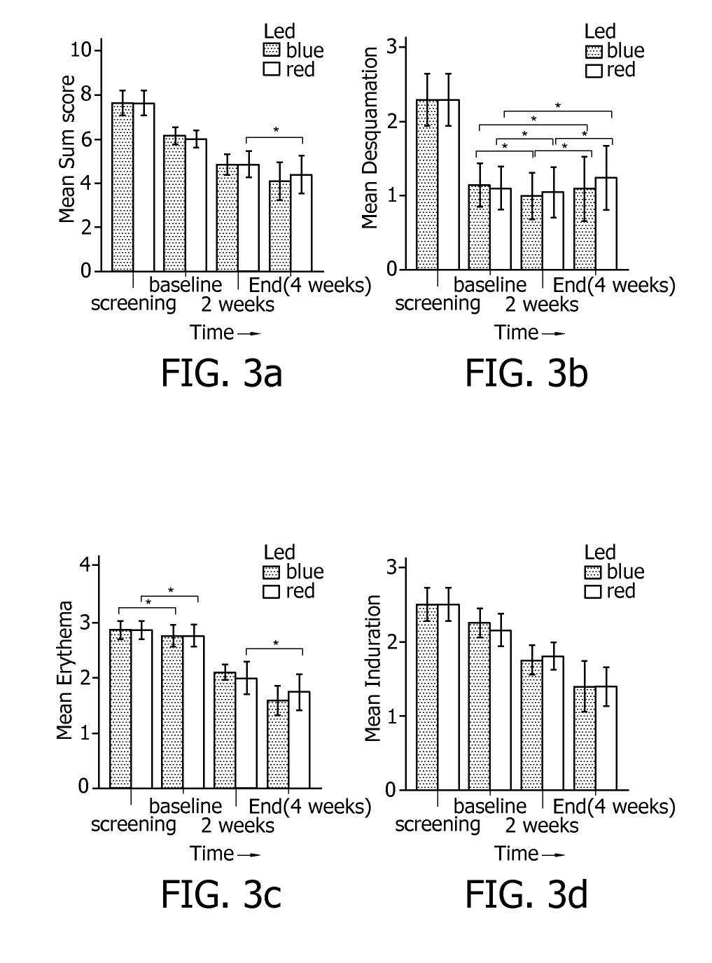

The patients were treated for 4 weeks, 3 times a week for 20 min (i.e. during a first period of time at regular intervals), and they were checked in the beginning, after 2 weeks, and after 4 weeks. The study was double blinded (=the patients didn't know which type of light was the right one, and the doctor didn't know which plaque was treated with which light). The results are shown in FIG. 3, where the clinical severity score of psoriasis plaques is shown. “A” is the total sum score, “B” shows the changes in desquamation (the dry dead skin cells), “C” the changes in erythema (how red is the skin), and “D” the changes in induration (the thickness of the plaque). The initial score, measured at the baseline (the beginning of the clinical trial), decreases over the 4 weeks of treatment. This reduction is statistically significant both for the blue and for the red. There is no statistically relevant difference between the red and the blue. This is a surprising result, since in the in vitro studies the red light irradiation doesn't show any influence on the cell division rate. Probably there are in vivo two factors, which might play a role and explain this unexpected result. In vivo the red light has influence on other things that just the skin cells, like maybe on the blood circulation. Furthermore, the red light penetrates deeper in the skin 5 than the blue light although red is more reflected by the skin than blue.

The present use embodiments of direct radiation treatment of psoriatic cells in human skin 5 may use several apparatus embodiment, as discussed below.

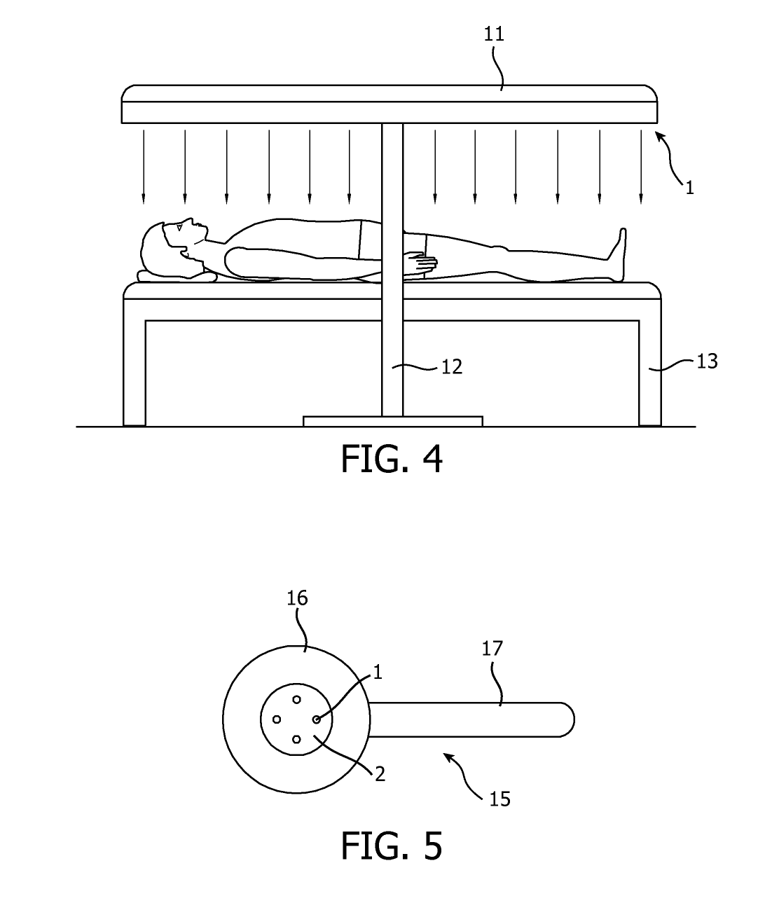

One embodiment of the invention is shown in FIG. 4. This is a half body device embodiment, wherein the apparatus 10 is positioned in an armature 11 in the form of an elongate holder held above a patient's body by a gantry type of structure, e.g. in the form of a mounting rack 12. In operation, the patient is lying on a bed 13 underneath the elongate holder 11. The mounting rack 12 is arranged to hold the armature 11 in an adjustable manner. The apparatus comprises a radiation source 1 in the elongate holder, e.g. with an array of blue LED's. A kind of cabin version, where the patient can have a full body treatment can also be made. Another version of this embodiment can be made with red LED's only, or with a combination of both blue LED's and red LED's. The latter embodiment may be very effective considering that red light penetrates deeper into the skin 5 (but is more reflected by the skin 5), and blue penetrates less deep (but is less reflected by the skin 5). This full body or half body treatment can be done at the hospital or at home.

A further embodiment of the present invention is shown in FIG. 5 where a relatively small, portable device 15 can be used to treat a patient's skin 5 with a mild type of psoriasis (just few small plaques). The device 15 comprises a head 16, wherein the apparatus 10 is mounted, e.g. using a printed circuit board as reflector 2 which is provided with a number of LED's as radiation source 1. Also this type of device 15 can be made with blue, red or red and blue LED's.

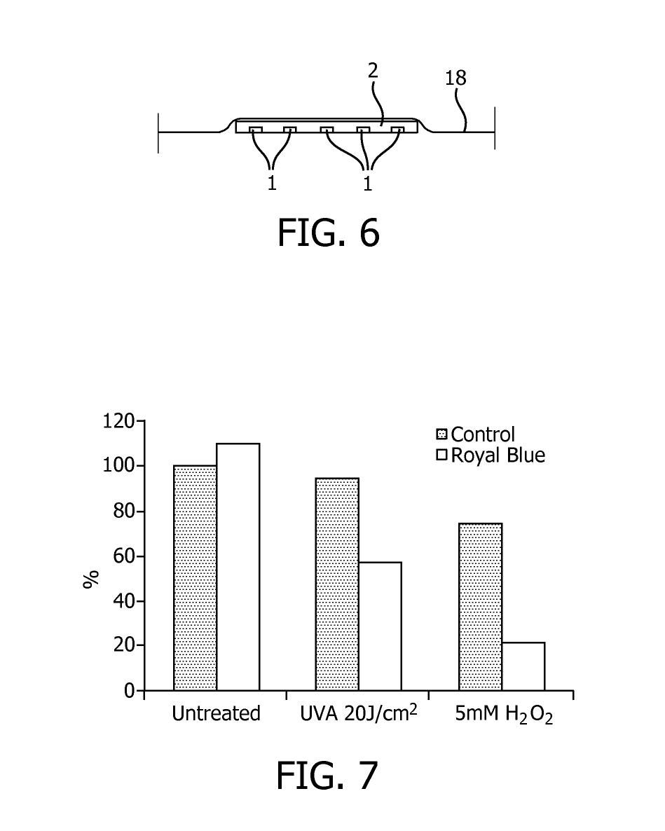

An even further embodiment of the present invention is a wearable solution, in the form of a plaster 18, as shown in FIG. 6. Also this one can be made with blue, red or red and blue LED's. The plaster 18 is provided with a very thin embodiment of the apparatus 10, e.g. using a (flexible) printed circuit board as reflector 2, and a plurality of LED's as radiation source 1.

For all the device embodiments described with reference to FIG. 4-6, the power density on the skin 5 can be controlled to be up to 200 mW/cm² (e.g. ˜100 mW/cm²) for the blue light radiation (which is more or less the maximum power density that the skin 5 can stand without becoming too warm), and up to 300 mW/cm² (e.g. ˜200 mW/cm²) for the red light radiation (which is less perceived by the heat sensors in the skin and therefore gives a too warm sensation at higher power densities). Furthermore, the various apparatus embodiments can be used to be as close as possible to the skin 5, in order to maximize the power density delivered to the skin 5.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

1. A treatment apparatus for treatment of parts of a skin, comprising:
a radiation source emitting radiation; and
a radiator for guiding the emitted radiation to the parts of the skin, wherein the parts of the skin comprise skin cells affected by psoriasis, and wherein the radiation source emits radiation in a first wavelength range of 400-460 nm, in a second wavelength range of 600-700 nm, or in a first wavelength range of 400-460 nm and a second wavelength range of 600-700 nm.

2. The treatment apparatus of claim 1, wherein the radiation source emits radiation with a wavelength of 420 nm.

3. The treatment apparatus of claim 1, wherein the radiator provides an energy in the first wavelength range to the affected parts of the skin of up to 200 mW/cm^{2.}

4. The treatment apparatus of claim 1, wherein the radiator provides an energy in the second wavelength range to the affected parts of the skin of up to 300 mW/cm^{2.}

5. The treatment apparatus of claim 1, wherein the radiation source is a LED based device.

6. The treatment apparatus of claim 1, further comprising an armature in which the radiation source and radiator are accommodated, and a mounting rack in which the armature is adjustably held.

7. The treatment apparatus of claim 1, further comprising a portable treatment head in which the radiation source and radiator are housed.

8. The treatment apparatus of claim 1, further comprising a plaster for attaching the treatment apparatus to the skin.

9. Use of a treatment apparatus comprising a radiation source emitting radiation and a radiator for guiding the emitted radiation for the treatment of psoriasis, comprising irradiating parts of a skin having skin cells affected by psoriasis wherein the radiation comprises radiation in a first wavelength range of 400-460 nm, in a second wavelength range of 600-700 nm, or in a first wavelength range of 400-460 nm and a second wavelength range of 600-700 nm.

10. Use according to claim 9, in combination with a Psoralen UVA therapy.

11. Use according to claim 9, in combination with a peroxide treatment or UV treatment.

12. Use according to claim 9, wherein the skin is irradiated during a first period of time, at predetermined intervals.
